(19) United States
(12) Patent Application Publication
(10) Pub. No.: US 2022/0160334 A1
(43) Pub. Date: May 26, 2022

(54) METHOD AND SYSTEM FOR ENHANCED VISUALIZATION OF A PLEURAL LINE BY AUTOMATICALLY DETECTING AND MARKING THE PLEURAL LINE IN IMAGES OF A LUNG ULTRASOUND SCAN

(71) Applicant: GE Precision Healthcare LLC, Wauwatosa, WI (US)

(72) Inventors: Rahul Venkataramani, Bangalore (IN); Dani Pinkovich, Atlit (IL)

(21) Appl. No.: 17/101,149

(22) Filed: Nov. 23, 2020

(51) Int. Cl. A61B 8/08; A61B 8/00

(52) U.S. Cl. CPC A61B 8/5276; A61B 8/5223; A61B 8/5246; A61B 8/461

(57) ABSTRACT

A system and method for enhancing visualization of a pleural line by automatically detecting and marking the pleural line in images of an ultrasound scan is provided. The method includes receiving an ultrasound cine loop acquired according to a first mode. The method includes processing the ultrasound cine loop according to the first mode. The method includes processing at least a portion of the ultrasound cine loop according to a second mode. The method includes identifying a position of an anatomical structure based on the at least a portion of the ultrasound cine loop processed according to the second mode. The method includes displaying, at a display system, the position of the anatomical structure on a first mode image generated from the ultrasound cine loop processed according to the first mode.

### FIELD

Certain embodiments relate to ultrasound imaging. More specifically, certain embodiments relate to a method and system for enhancing visualization of a pleural line in lung ultrasound images by automatically detecting and marking the pleural line in images of a lung ultrasound scan.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

Ultrasound imaging is inexpensive, portable, and exhibits comparatively lesser risk of COVID-19 transmission compared to other image modalities, such as computed tomography (CT), X-ray, and the like. Ultrasound imaging is also known to be sensitive to detecting many lung abnormalities. Ultrasound images may provide various indications useful in identifying COVID-19. For example, a normal pleural region depicted in B-mode ultrasound images may be a thin, bright, consistent line. Common COVID-19 signatures, however, may depict the pleural line as non-continuous and/or wide (i.e., thickened pleural) in B-mode ultrasound images. Automated pleural detection in B-mode ultrasound images typically involves the analysis of an entire video sequence, which is computationally expensive and time-consuming.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for enhancing visualization of a pleural line by automatically detecting and marking the pleural line in images of an ultrasound scan, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to provide enhanced visualization of a pleural line by automatically detecting and marking the pleural line in images of an ultrasound scan, in accordance with various embodiments.

FIG. 2 illustrates screenshots of an exemplary M-mode ultrasound image and a corresponding enhanced B-mode ultrasound image of a portion of a lung having a marker identifying a pleural line, in accordance with various embodiments.

FIG. 3 is a flow chart illustrating exemplary steps that may be utilized for providing enhanced visualization of a pleural line by automatically detecting and marking the pleural line in images of an ultrasound scan, in accordance with various embodiments.

### DETAILED DESCRIPTION

Certain embodiments may be found in a method and system for enhancing visualization of a pleural line by automatically detecting and marking the pleural line in images of an ultrasound scan. For example, aspects of the present disclosure have the technical effect of automatically providing real-time or stored ultrasound images enhanced to identify the pleural line for presentation to an ultrasound operator. Moreover, aspects of the present disclosure have the technical effect of reducing computation time and resources by automatically marking a pleural line in B-mode images generated from an acquired cine loop based on identification of the pleural line in a limited number of M-mode images (e.g., 1-3 M-mode images). Furthermore, aspects of the present disclosure are more tolerant to noise and other artifacts in image acquisition because M-mode image(s) are processed to identify the pleural line instead of the B-mode images. Additionally, aspects of the present disclosure have the technical effect of simplifying post-processing to detect COVID-19 signatures, such as pleural irregularity, by detecting the pleural line in M-mode image(s) and marking the pleural line in B-mode images.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, Contrast Enhanced Ultrasound (CEUS), and/or sub-modes of B-mode and/or CF such as Harmonic Imaging, Shear Wave Elasticity Imaging (SWEI), Strain Elastography, TVI, PDI, B-flow, MVI, UGAP, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), DSP, FPGA, ASIC or a combination thereof.

Additionally, the term pleural line, as used herein, refers to the pleura and/or pleural region depicted in the ultrasound image data. Although certain embodiments may describe detection of a pleural line in M-mode image(s) and marking the pleural line in B-mode image(s), for example, unless so claimed, the scope of various aspects of the present invention should not be limited to a pleural line, M-mode images, and B-mode images and may additionally and/or alternatively be applicable to any suitable anatomical structures and imaging modes.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

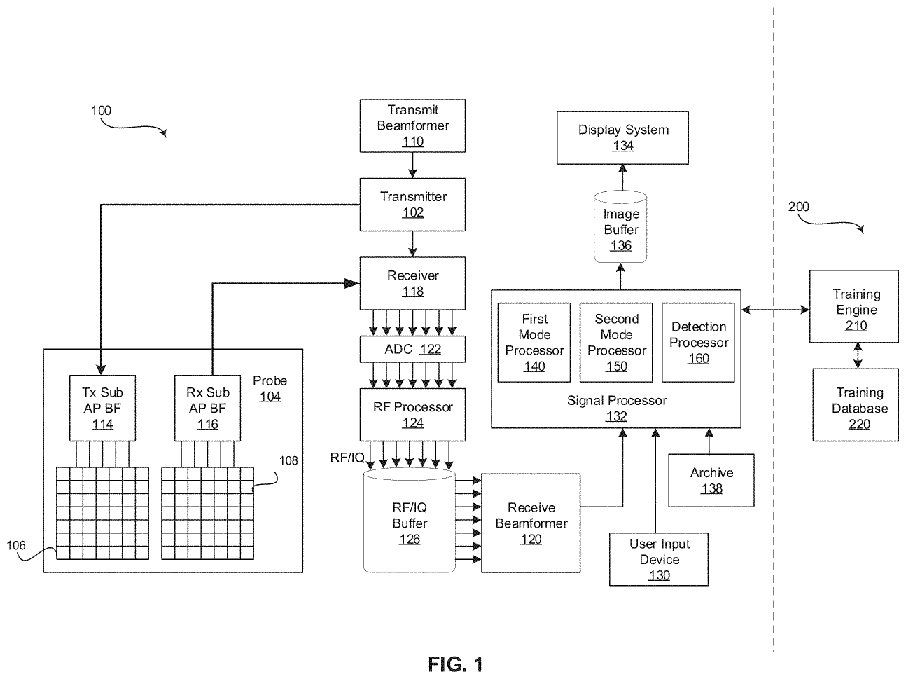

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to provide enhanced visualization of a pleural line by automatically detecting and marking the pleural line in images of an ultrasound scan, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two-dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as a lung, a fetus, a heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or a plurality of A/D converters 122.

0 The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, image acquisition and scan parameters, settings, configuration parameters, select protocols and/or templates, change scan mode, manipulate tools for reviewing acquired ultrasound data, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, graphic processing units, microprocessors, microcontrollers, and/or the like. The signal processor 132 may be an integrated component, or may be distributed across various locations, for example. In an exemplary embodiment, the signal processor 132 may comprise a first mode processor 140, a second mode processor 150, and a detection processor 160 and may be capable of receiving input information from a user input device 130 and/or archive 138, generating an output displayable by a display system 134, and manipulating the output in response to input information from a user input device 130, among other things. The signal processor 132, first mode processor 140, second mode processor 150, and detection processor 160 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a first mode processor 140 that comprises suitable logic, circuitry, interfaces and/or code that may be operable to process acquired and/or retrieved ultrasound image data to generate ultrasound images according to a first mode. For example, the first mode may be a B-mode and the first mode processor 140 may be configured to process a received cine loop of ultrasound data into B-mode frames.

In various embodiments, the first mode processor 140 comprises suitable logic, circuitry, interfaces and/or code that may be operable to perform further image processing functionality, such as detecting rib shadows in a B-mode lung ultrasound image. For example, the first mode processor 140 may detect rib shadows by executing image recognition algorithms, artificial intelligence, and/or any suitable image recognition technique. As an example, the first mode processor 140 may deploy deep neural network(s) (e.g., artificial intelligence model(s)) that may be made up of, for example, an input layer, an output layer, and one or more hidden layers in between the input and output layers. Each of the layers may be made up of a plurality of processing nodes that may be referred to as neurons. For example, the first mode processor 140 may inference an artificial intelligence model comprising an input layer having a neuron for each pixel or a group of pixels from a scan plane of an anatomy. The output layer may have neurons corresponding to one or more features of the imaged anatomy. As an example, the output layer may identify rib shadows and/or any suitable imaged anatomy features. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the first mode processor 140 inferencing the deep neural network (e.g., convolutional neural network) may identify rib shadows in B-mode ultrasound images with a high degree of probability. The locations of detected rib shadows may be provided to the second mode processor 150 and/or may be stored at archive 138 or any suitable data storage medium.

The signal processor 132 may include a second mode processor 150 that comprises suitable logic, circuitry, interfaces and/or code that may be operable to process a portion of the acquired and/or retrieved ultrasound image data to generate ultrasound images according to a second mode. For example, the second mode may be an M-mode and the second mode processor 150 may be configured to process a portion of a received cine loop of ultrasound data into one or more M-mode images. In a representative embodiment, the second mode processor 150 may be configured to generate 1-3 M-mode images from the cine loop. The M-mode images each correspond to one location (i.e., line) in the B-mode images over time. As an example, a cine loop of ultrasound data of a lung may be acquired over a period of time, such as one or more breathing cycles. For example, the cine loop of ultrasound data may correspond with 100 B-mode frames or any suitable number of B-mode frames. Each of the B-mode frames may include a number of lines of ultrasound data, such as 160 lines or any suitable number of lines of ultrasound data. The second mode processor 150 may be configured to generate an M-mode image from one (1) of the 160 lines at a same location in each of the 100 B-mode frames. In certain embodiments, a virtual M-mode line may be overlaid on a displayed B-mode image to illustrate a location of a simultaneously displayed M-mode image. In an exemplary embodiment, the second mode processor 150 selects one or more locations (i.e., virtual M-mode line positions) in the B-mode images to generate the one or more M-mode images. The selection of the one or more locations in the B-mode image may correspond with default locations and/or may be based on rib shadow locations as detected by the first mode processor 140. As an example, the second mode processor 150 may be configured to select one or more locations (i.e., virtual M-mode line positions) that do not include rib shadows. The M-mode images (e.g., 1-3 M-mode images) generated by the second mode processor 150 may be provided to the detection processor 160 and/or may be stored at archive 138 or any suitable data storage medium.

The signal processor 132 may include a detection processor 160 that comprises suitable logic, circuitry, interfaces and/or code that may be operable to identify a position of an anatomical structure based on the portion of the ultrasound image data processed according to the second mode. For example, the detection processor 160 may be configured to automatically detect a pleural line depicted in the M-mode image(s) generated by the second mode processor 150. The anatomical structure identification may be performed by the detection processor 160 executing image recognition algorithms, artificial intelligence, and/or any suitable image recognition technique. For example, the detection processor 160 may perform feature extraction to generate a histogram of orientation gradients corresponding to the M-mode image. The detection processor 160 may employ separation logic to determine a pleural line depicted in the M-mode image based on the generated histogram of orientation gradients (e.g., an average top edge and average bottom edge of the pleura).

As another example, the detection processor 160 may deploy deep neural network(s) (e.g., artificial intelligence model(s)) that may be made up of, for example, an input layer, an output layer, and one or more hidden layers in between the input and output layers. Each of the layers may be made up of a plurality of processing nodes that may be referred to as neurons. For example, the detection processor 160 may inference an artificial intelligence model comprising an input layer having a neuron for each pixel or a group of pixels from second mode image (e.g., an M-mode image). The output layer may have neurons corresponding to one or more anatomical structures, such as a pleural line. As an example, the output layer may identifying a pleural line, and/or any suitable anatomical structure in the M-mode image. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the detection processor 160 inferencing the deep neural network (e.g., convolutional neural network) may identifying a pleural line in the second mode image with a high degree of probability.

The detection processor 160 may comprise suitable logic, circuitry, interfaces and/or code that may be operable to mark, in the generated first mode images, the anatomical structure detected in the second mode images. For example, the markings may include lines, a box, colored highlighting, labels, and the like overlaid on the first mode images. In various embodiments, the detection processor 160 may be configured to colorize pixels of the first mode image to provide the markers. The marked first mode image(s) identifying the detected anatomical structure may be presented to a user at the display system 134, stored at archive 138 or any suitable data storage medium, and/or provided to signal processor 132 for further image analysis and/or processing. As an example, B-mode images including markers identifying the pleural line may be presented at display system 132, stored at archive 138 or any suitable data storage medium, and/or further processed by the signal processor 132 to detect COVID-19 specific signatures, such as pleura irregularity and the like.

The detection of the pleural line in the limited number of M-mode images (e.g., 1-3 M-mode images) for marking the pleural line in the B-mode images as performed by the detection processor 160 reduces computational resources and computation time compared to the processing of the B-mode frames of a cine loop (e.g., 100 B-mode frames) to detect and mark the pleural line. The detection of the pleural line in the limited number of M-mode images for marking the pleural line in the B-mode images as performed by the detection processor is also more tolerant to noise and other artifacts in image acquisition compared to the processing of the B-mode frames of a cine loop to detect and mark the pleural line.

In an exemplary embodiment, the first mode images (e.g., B-mode frames) having the markings identifying the anatomical structure (e.g., pleural line) may be dynamically presented at a display system 134 such that an operator of the ultrasound probe 104 may view the marked images in substantially real-time. The B-mode images highlighted by the detection processor 160 may be stored at the archive 138. The archive 138 may be a local archive, a Picture Archiving and Communication System (PACS), or any suitable device for storing ultrasound images and related information.

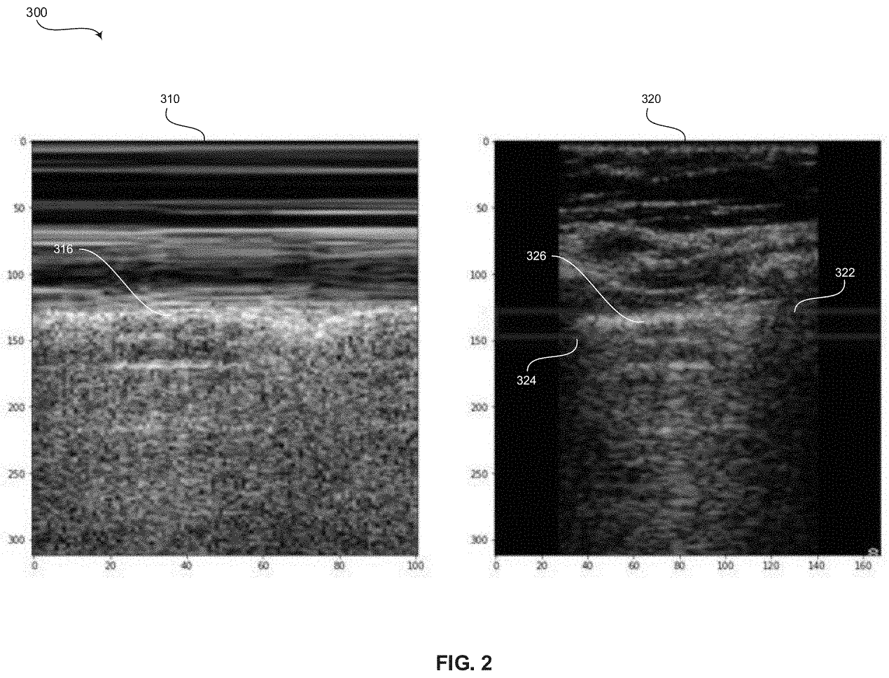

FIG. 2 illustrates screenshots 300 of an exemplary M-mode ultrasound image 310 and a corresponding enhanced B-mode ultrasound image 320 of a portion of a lung having a marker 322, 324 identifying a pleural line 326, in accordance with various embodiments. Referring to FIG. 2, screenshots 300 of an M-mode image 310 and B-mode image 320 of a lung are shown having a pleura line 316, 326 extending generally horizontal. In an exemplary embodiment, the M-mode image 310 may be generated by the second mode processor 150 at a location in the B-mode images 320 based at least in part on a location of detected ribs (not shown), which may be recognized in the B-mode images 320 by their acoustic shadow. The detection processor 160 may search the M-mode image 310 for the bright horizontal section that identifies the pleura 316. The detection processor 160 may mark 322, 324 the pleural line 326 in the B-mode images 320 based on the detection of the pleural line 316 in the M-mode image 310. The markings 322, 324 in the B-mode images 320 may be a line 322 identifying an average top edge of the pleural line 326 and a line 324 identifying an average bottom edge of the pleural line 326. Additionally and/or alternatively, the markings 322, 324 in the B-mode images 320 may include identifiers (e.g., arrows, circles, squares, stars, etc.) at the outer side or sides of the B-mode image 320 identifying the top and bottom edges of the pleural line 326, a box in the B-mode images 320 surrounding the pleural line 326, colored highlighting of the pleural line 326, labeling of the pleural line 326, and the like overlaid on the B-mode images 320. In various embodiments, the detection processor 160 may be configured to colorize pixels of the pleural line 326 in the B-mode images 320.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present B-mode ultrasound images 320 with markings 322, 324 identifying a pleural line 326, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores first mode images (e.g., B-mode images 320), first mode images having markings 322, 324, second mode images (e.g., M-mode images 310), instructions for processing received ultrasound image data according to a first mode, instructions for processing received ultrasound image data according to a second mode, instructions for detecting anatomical structures (e.g., pleural line 316) in a second mode image 310 and marking 322, 324 the anatomical structures (e.g., pleural line 326) in a first mode image 320, instructions for detecting anatomical features (e.g., rib shadows) in a first mode image 320, and/or artificial intelligence models deployable to perform anatomical structure and/or feature detection, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 160 may comprise suitable logic, circuitry, interfaces and/or code that may be operable to train the neurons of the deep neural network(s) (e.g., artificial intelligence model(s)) inferenced (i.e., deployed) by the first mode processor 140 and/or the detection processor 160. For example, the artificial intelligence model inferenced by the first mode processor 140 may be trained to automatically identify anatomical features (e.g., rib shadows) in first mode images (e.g., B-mode images 320). As an example, the training engine 210 may train the deep neural networks deployed by the first mode processor 140 using database(s) 220 of classified ultrasound images of various anatomical features. The ultrasound images may include first mode ultrasound images of a particular anatomical feature, such as B-mode images 320 having rib shadows, or any suitable ultrasound images and features. As another example, the artificial intelligence model inferenced by the detection processor 160 may be trained to automatically identify anatomical structure (e.g., a pleural line 316) in second mode images (e.g., M-mode images 310). As an example, the training engine 210 may train the deep neural networks deployed by the detection processor 160 using database(s) 220 of classified ultrasound images of various anatomical structures. The ultrasound images may include second mode ultrasound images of a particular anatomical structure, such as M-mode images 310 having a pleural line 316, or any suitable ultrasound images and structures.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms.

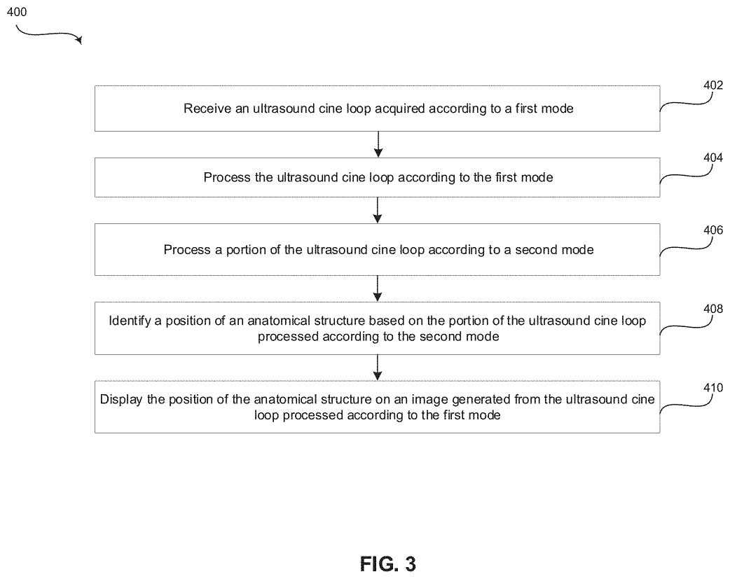

FIG. 3 is a flow chart 400 illustrating exemplary steps that may be utilized for providing enhanced visualization of a pleural line 326 by automatically detecting and marking the pleural line 326 in images 320 of an ultrasound scan, in accordance with various embodiments. Referring to FIG. 3, there is shown a flow chart 400 comprising exemplary steps 402 through 410. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 402, a signal processor 132 of an ultrasound system 100 or a remote workstation may receive an ultrasound cine loop acquired according to a first mode. For example, an ultrasound probe 104 in the ultrasound system 100 may be operable to perform an ultrasound scan of a region of interest, such as a zone of a lung. The ultrasound scan may be performed according to the first mode, such as a B-mode or any suitable image acquisition mode. An ultrasound operator may acquire an ultrasound cine loop having a plurality of frames. The ultrasound scan may be acquired, for example, over the duration of at least one breathing cycle. The breathing cycle can be detected automatically, by a specified duration, or by an operator, among other things. For example, if a patient is using a ventilator, the ventilator can provide a signal to the signal processor 132 identifying the breathing cycle duration. As another example, the breathing cycle may be defined by an operator input at a user input module 130 or be a default value, such as 3-5 seconds. Further, an operator may identify the end of a breathing cycle by providing an input at the user input module 130, such as by pressing a button on the ultrasound probe 104. The ultrasound cine loop may be received by the signal processor 132 and/or stored to archive 138 or any suitable data storage medium from which the signal processor 132 may retrieve the cine loop.

At step 404, the signal processor 132 may process the ultrasound cine loop according to the first mode. For example, the first mode may be a B-mode and a first mode processor 140 of the signal processor 132 may be configured to process a received cine loop of ultrasound data into B-mode frames 320. In various embodiments, the first mode processor 140 may be configured to perform further image processing functionality, such as detecting rib shadows in a B-mode lung ultrasound image 320. As an example, the first mode processor 140 may detect rib shadows by executing image recognition algorithms, artificial intelligence, and/or any suitable image recognition technique.

At step 406, the signal processor 132 may process a portion of the ultrasound cine loop according to a second mode. For example, the second mode may be an M-mode and a second mode processor 150 of the signal processor 132 may be configured to process a portion of the received cine loop of ultrasound data into one or more M-mode images 310. In an exemplary embodiment, the second mode processor 150 may be configured to generate 1-3 M-mode images 310 from the cine loop. The 1-3 M-mode images 310 may correspond to 1-3 locations selected by the second mode processor 150 in the B-mode images 320. The selection of the one or more locations in the B-mode image may correspond with default locations and/or may be based on rib shadow locations as detected by the first mode processor 140.

At step 408, the signal processor 132 may identify a position of an anatomical structure 316 based on the portion of the ultrasound cine loop processed according to the second mode. For example, the detection processor 160 may be configured to automatically detect a pleural line 316, or any suitable anatomical structure, depicted in the M-mode image(s) 310, or any suitable second mode image(s), generated by the second mode processor 150. The anatomical structure identification may be performed by the detection processor 160 executing image recognition algorithms, artificial intelligence, and/or any suitable image recognition technique. For example, the detection processor 160 may perform feature extraction to generate a histogram of orientation gradients corresponding to the M-mode image 310. The detection processor 160 may employ separation logic to determine a pleural line 316 depicted in the M-mode image 310 based on the generated histogram of orientation gradients. As another example, the detection processor 160 may deploy deep neural network(s) (e.g., artificial intelligence model(s)) that may identify an anatomical structure (e.g., pleural line 316) in the second mode image (e.g., M-mode image 310) with a high degree of probability.

At step 410, the signal processor 132 may display the position of the anatomical structure on an image 320 generated from the ultrasound cine loop processed according to the first mode. For example, the detection processor 160 may be configured to mark 322, 324, in the generated first mode images 320, the anatomical structure 316, 326 detected in the second mode images 310. The markings may include lines 322, 324, a box, colored highlighting, labels, and the like overlaid on the first mode images 320. Additionally and/or alternatively, the detection processor 160 may be configured to colorize pixels of the first mode images 320 to provide the markers 322, 324. The marked first mode image(s) (e.g., B-mode images 320) identifying the detected anatomical structure (e.g., pleural line 326) may be presented to a user at the display system 134. In a representative embodiment, the first mode images 320 may be further processed by the signal processor 132 to detect COVID-19 specific signatures, such as pleura irregularity and the like. The processing of the first mode images 320 by the signal processor 132 may include, for example, executing image recognition algorithms, artificial intelligence, and/or any suitable image recognition technique to detect non-continuous and/or wide pleural lines 326 in B-mode images 320.

Aspects of the present disclosure provide a method 400 and system 100 for enhancing visualization of a pleural line 326 by automatically detecting and marking 322, 324 the pleural line 316, 326 in images 310, 320 of an ultrasound scan. In accordance with various embodiments, the method 400 may comprise receiving 402, by at least one processor 132, 140, 150, an ultrasound cine loop acquired according to a first mode. The method 400 may comprise processing 404, by the at least one processor 132, 140, the ultrasound cine loop according to the first mode. The method 400 may comprise processing 406, by the at least one processor 132, 150, at least a portion of the ultrasound cine loop according to a second mode. The method 400 may comprise identifying 408, by the at least one processor 132, 160, a position of an anatomical structure 316 based on the at least a portion of the ultrasound cine loop processed according to the second mode. The method 400 may comprise displaying 410, by the at least one processor 132, 140, 160 at a display system 132, the position 322, 324 of the anatomical structure 326 on a first mode image 320 generated from the ultrasound cine loop processed according to the first mode.

In an exemplary embodiment, the first mode may be a B-mode. In a representative embodiments, the second mode may be an M-mode. In various embodiments, the processing 404 the ultrasound cine loop according to the first mode may comprise generating B-mode images 320 and detecting rib shadows in the B-mode images 320. The processing 406 the at least the portion of the ultrasound cine loop according to the second mode may comprise generating at least one M-mode image 310 based on the detected rib shadows in the B-mode images 320. In certain embodiments, the processing 406 the at least a portion of the ultrasound cine loop according to the second mode may comprise generating 1-3 M-mode images 310. In an exemplary embodiment, the anatomical structure may be a pleural line 316, 326. In a representative embodiment, the identifying 408 the position of the anatomical structure 316 may comprise performing feature extraction by generating a histogram of oriented gradients, and employing separation logic to determine the anatomical structure 316 depicted in a second mode image 310 based on the histogram of orientation gradients. The second mode image 310 may be generated from the at least the portion of the ultrasound cine loop according to the second mode.

Various embodiments provide a system 100 for enhancing visualization of a pleural line 326 by automatically detecting and marking 322, 324 the pleural line 316, 326 in images 310, 320 of an ultrasound scan. The ultrasound system 100 may comprise at least one processor 132, 140, 150, 160 and a display system 134. The at least one processor 132, 140 may be configured to receive an ultrasound cine loop acquired according to a first mode. The at least one processor 132, 140 may be configured to process the ultrasound cine loop according to the first mode. The at least one processor 132, 150 may be configured to process at least a portion of the ultrasound cine loop according to a second mode. The at least one processor 132, 160 may be configured to identify a position of an anatomical structure 316 based on the at least a portion of the ultrasound cine loop processed according to the second mode. The display system 134 may be configured to display the position 322, 324 of the anatomical structure 326 on a first mode image 320 generated from the ultrasound cine loop processed according to the first mode.

In a representative embodiment, the first mode may be a B-mode. In various embodiments, the second mode may be an M-mode. In certain embodiments, the at least one processor 132, 140 may be configured to process the ultrasound cine loop according to the first mode by generating B-mode images 320 and detecting rib shadows in the B-mode images 320. The at least one processor 132, 150 may be configured to process the at least the portion of the ultrasound cine loop according to the second mode by generating at least one M-mode image 310 based on the detected rib shadows in the B-mode images 320. In an exemplary embodiment, the at least one processor 132, 150 may be configured to process the at least a portion of the ultrasound cine loop according to the second mode to generate 1-3 M-mode images 310. In a representative embodiment, the anatomical structure may be a pleural line 316, 326. In various embodiments, the at least one processor 132, 160 may be configured to identify the position of the anatomical structure 316 by performing feature extraction by generating a histogram of oriented gradients, and employing separation logic to determine the anatomical structure 316 depicted in a second mode image 310 based on the histogram of orientation gradients. The second mode image 310 may be generated from the at least the portion of the ultrasound cine loop according to the second mode.

Certain embodiments provide a non-transitory computer readable medium having stored thereon, a computer program having at least one code section. The at least one code section is executable by a machine for causing the machine to perform steps 400. The steps 400 may comprise receiving 402 an ultrasound cine loop acquired according to a first mode. The steps 400 may comprise processing 404 the ultrasound cine loop according to the first mode. The steps 400 may comprise processing 406 at least a portion of the ultrasound cine loop according to a second mode. The steps 400 may comprise identifying 408 a position of an anatomical structure 316 based on the at least a portion of the ultrasound cine loop processed according to the second mode. The steps 400 may comprise displaying 410 the position 322, 324 of the anatomical structure 326 on a first mode image 320 generated from the ultrasound cine loop processed according to the first mode at a display system 132.

In various embodiments, the first mode is B-mode and the second mode is M-mode. In certain embodiments, the processing the ultrasound cine loop according to the first mode may comprise generating B-mode images 320 and detecting rib shadows in the B-mode images 320. The processing the at least the portion of the ultrasound cine loop according to the second mode may comprise generating at least one M-mode image 310 based on the detected rib shadows in the B-mode images 320. In an exemplary embodiment, the processing the at least a portion of the ultrasound cine loop according to the second mode comprises generating 1-3 M-mode images 310. In a representative embodiment, the anatomical structure is a pleural line 316, 326. In various embodiments, the identifying the position of the anatomical structure may comprise performing feature extraction by generating a histogram of oriented gradients and employing separation logic to determine the anatomical structure 316 depicted in a second mode image 310 based on the histogram of orientation gradients. The second mode image 310 may be generated from the at least the portion of the ultrasound cine loop according to the second mode.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for enhancing visualization of a pleural line by automatically detecting and marking the pleural line in images of an ultrasound scan.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method, comprising:
receiving, by at least one processor, an ultrasound cine loop acquired according to a first mode;
processing, by the at least one processor, the ultrasound cine loop according to the first mode;
processing, by the at least one processor, at least a portion of the ultrasound cine loop according to a second mode;
identifying, by the at least one processor, a position of an anatomical structure based on the at least a portion of the ultrasound cine loop processed according to the second mode; and
displaying, by the at least one processor at a display system, the position of the anatomical structure on a first mode image generated from the ultrasound cine loop processed according to the first mode.

2. The method of claim 1, wherein the first mode is B-mode.

3. The method of claim 2, wherein the second mode is M-mode.

4. The method of claim 2, wherein the processing the ultrasound cine loop according to the first mode comprises:
generating B-mode images; and
detecting rib shadows in the B-mode images; and
wherein the processing the at least the portion of the ultrasound cine loop according to the second mode comprises generating at least one M-mode image based on the detected rib shadows in the B-mode images.

5. The method of claim 3, wherein the processing the at least a portion of the ultrasound cine loop according to the second mode comprises generating 1-3 M-mode images.

6. The method of claim 1, wherein the anatomical structure is a pleural line.

7. The method of claim 1, wherein the identifying the position of the anatomical structure comprises:
performing feature extraction by generating a histogram of oriented gradients; and
employing separation logic to determine the anatomical structure depicted in a second mode image based on the histogram of orientation gradients, the second mode image generated from the at least the portion of the ultrasound cine loop according to the second mode.

8. A system, comprising:
at least one processor configured to:
receive an ultrasound cine loop acquired according to a first mode;
process the ultrasound cine loop according to the first mode;
process at least a portion of the ultrasound cine loop according to a second mode; and
identify a position of an anatomical structure based on the at least a portion of the ultrasound cine loop processed according to the second mode; and
a display system configured to display the position of the anatomical structure on a first mode image generated from the ultrasound cine loop processed according to the first mode.

9. The system of claim 8, wherein the first mode is B-mode.

10. The system of claim 9, wherein the second mode is M-mode.

11. The system of claim 9, wherein the at least one processor is configured to process the ultrasound cine loop according to the first mode by:
generating B-mode images; and
detecting rib shadows in the B-mode images; and
wherein the at least one processor is configured to process the at least the portion of the ultrasound cine loop according to the second mode by generating at least one M-mode image based on the detected rib shadows in the B-mode images.

12. The system of claim 10, wherein the at least one processor is configured to process the at least a portion of the ultrasound cine loop according to the second mode to generate 1-3 M-mode images.

13. The system of claim 8, wherein the anatomical structure is a pleural line.

14. The system of claim 8, wherein the at least one processor is configured to identify the position of the anatomical structure by:
performing feature extraction by generating a histogram of oriented gradients; and
employing separation logic to determine the anatomical structure depicted in a second mode image based on the histogram of orientation gradients, the second mode image generated from the at least the portion of the ultrasound cine loop according to the second mode.

15. A non-transitory computer readable medium having stored thereon, a computer program having at least one code section, the at least one code section being executable by a machine for causing the machine to perform steps comprising:
receiving an ultrasound cine loop acquired according to a first mode;
processing the ultrasound cine loop according to the first mode;
processing at least a portion of the ultrasound cine loop according to a second mode;
identifying a position of an anatomical structure based on the at least a portion of the ultrasound cine loop processed according to the second mode; and
displaying the position of the anatomical structure on a first mode image generated from the ultrasound cine loop processed according to the first mode at a display system.

16. The non-transitory computer readable medium of claim 15, wherein the first mode is B-mode and the second mode is M-mode.

17. The non-transitory computer readable medium of claim 16, wherein the processing the ultrasound cine loop according to the first mode comprises:
generating B-mode images; and
detecting rib shadows in the B-mode images; and
wherein the processing the at least the portion of the ultrasound cine loop according to the second mode comprises generating at least one M-mode image based on the detected rib shadows in the B-mode images.

18. The non-transitory computer readable medium of claim 16, wherein the processing the at least a portion of the ultrasound cine loop according to the second mode comprises generating 1-3 M-mode images.

19. The non-transitory computer readable medium of claim 15, wherein the anatomical structure is a pleural line.

20. The non-transitory computer readable medium of claim 15, wherein the identifying the position of the anatomical structure comprises:
performing feature extraction by generating a histogram of oriented gradients; and
employing separation logic to determine the anatomical structure depicted in a second mode image based on the histogram of orientation gradients, the second mode image generated from the at least the portion of the ultrasound cine loop according to the second mode.
